(19) United States
(12) Patent Application Publication
(10) Pub. No.: US 2008/0289991 A1
(43) Pub. Date: Nov. 27, 2008

(54) Disposable Safety "Patient Kit" for Medical Devices

(76) Inventor: Fabio Sgarabottolo, Cervarese S. Croce (IT)

Correspondence Address: Themis Intellectual Property Counsel, 7660 Fay Ave Ste H535, La Jolla, CA, 92037, US

(21) Appl. No.: 12/094,986

(22) PCT Filed: Dec. 6, 2005
(86) PCT No.: PCT/IT05/00717

§ 371 Date: Jun. 6, 2008

(51) Int. Cl. A61B 19/02

(52) U.S. Cl. 206/569

(57) ABSTRACT

The invention concerns a new disposable package for test tubes, vials (O) and/or other medical devices for use by hospitals or other health facilities, comprising a container (C) provided with an aperture (A) for the insertion of said test tubes, vials (O) and/or medical devices in general, and at least one element (F) for closing and/or sealing said aperture (A), and wherein said element (F) is provided with the identification data (S) of the patient, etc. Said closing and identification element (F) is an adhesive sheet with tab (L), on which said patient identification data (S) can be provided, together with one or more incisions or perforation (P) and/or other systems for tear-opening of at least a part (F1) of said sheet (F). According to the invention an additional cover (R) can be used.

The present patent relates to containers used in hospitals, analysis laboratories, testing points or treatment centres in general and in particular concerns a new sealed disposable safety patient kit with container for test tubes, vials or other medical devices.

The use of test tubes, vials or containers of other types in medical-hospital environments for the collection of body fluids and biological samples taken from the patient for the purposes of analysis is known.

The organic sample to be analysed is placed in said test tubes or vials when it is taken from the patient.

Subsequently, each of said test tubes or vials, designed to contain the sample to be analysed, is marked with one or more labels indicating, for example, the name and/or identification code of the patient and/or the type of analysis to be performed on the sample taken and/or the origin and/or any other information necessary for the laboratory and the person taking the sample.

The test tubes and/or vials are labelled before the sample is taken, so that the medical or nursing staff taking the sample already have the exact number of test tubes and/or vials to be used, each showing precise indications concerning the sample to be taken, and further labels necessary to identify other medical equipment or devices for sampling.

After the sample has been taken, said test tubes and/or vials, or other containers designed to contain the samples for analysis, are sent to the analysis laboratory.

If the purposely printed identification labels were applied manually to the test tube or vial after the sample has been taken, there would be the risk of confusing the test tubes or vials of different patients or different types, therefore causing serious errors, with possible consequences for the health of the patient and/or delays in performance of the analysis or the beginning of appropriate treatment, with the need to repeat the test.

On the other hand, it is not infrequent for the test tubes or vials marked manually prior to the test to be confused, with consequent errors and also delays in performance of the analysis and communication of the results.

It is of fundamental importance for the health of the patients that for each of said patients tested, appropriate devices be used, correctly selected and labelled and with long-term batch traceability.

The subject of the present invention is a new sealed disposable safety patient kit with container for test tubes and/or vials or other medical devices, for use by hospitals, analysis laboratories, testing points or treatment centres in general.

The main aim of the present invention is to contain the test tubes and/or vials and/or other medical devices and/or other auxiliary labels of one single patient, distinguishing them and separating them from those of other patients.

A further aim of the present invention is to prevent confusion between test tubes and/or vials of different patients.

A further aim of the present invention is to prevent loss of the test tubes and/or vials and/or material tested.

A further aim of the present invention is to prevent deliberate malicious exchange or stealing or tampering with the test tubes and/or vials without leaving evidence of these operations.

These and other aims, direct and complementary, are achieved by the new sealed disposable safety patient kit with container for test tubes and/or vials or other medical devices, for use by hospitals or for treatment centres in general, with related adhesive closing and identification sheet.

The container is made preferably of plastic and comprises one or more apertures for insertion of the objects to be contained.

In the preferred embodiment of the invention, said container has a parallelepiped shape, with aperture obtained on its upper wall.

The test tubes and/or vials, in turn appropriately marked, and/or auxiliary labels and/or the other medical devices are placed inside said container. Said aperture of the container is then closed and sealed by means of a closing element or sheet made for example of paper, plastic-coated paper or plastic.

Said closing element can be for example an adhesive or partially adhesive sheet, i.e. provided with a layer of adhesive distributed at least on the surface in contact with said container.

For example, said element or sheet is adhesive only at the edges, where it comes into contact with the flat edges of said aperture of the container. This prevents malicious or accidental outflow of the material contained in the container.

The name and/or the identification codes of the patient and/or the type and quantity of samples to be taken and/or the date on which the sample was taken and/or the test centre and/or any other information useful for the medical, nursing or technical staff are furthermore printed on said closing element.

The container, prepared as described above, is delivered to the patient or to the medical, technical or nursing staff, who use it in the hospital department or at the place where the sample is taken (domicile of the patient within or outside the facility) or in the test centres or analysis laboratories.

In this way, there is absolutely no risk of losing the test tubes and/or vials, or of prescribing inappropriate treatment for the patient as a result of incorrect diagnoses caused by errors in the pre-testing phase.

The procedure for depositing the test tubes and/or vials or other medical devices inside the container can be performed manually or, preferably, via the use of automatic devices.

For example, said container, moving on a sliding belt, can stop at a first station where the test tubes and/or vials and/or auxiliary labels and/or other medical devices necessary for taking the sample are deposited inside the container.

Subsequently, the container in question stops at a station in which said closing element, printed with all the above-mentioned information, is applied to the aperture of the container for closing and identification purposes.

Said closing element furthermore comprises a protruding tab and one or more incisions or perforations, so that said sealed container can be opened by tearing, for use of the material contained.

It is also possible to print on said tab the identification data of the patient or of the material contained in the container.

The new patient kit can also be used as a safety element after the sample has been taken, during subsequent transport to the analysis laboratories.

For said purpose, according to the invention a further cover is used, separate from or integral with the container and applied to the container, to seal it, after said element or closing and identification sheet has been torn open.

Said cover is of the perimetric seal type, with perimetric teeth that snap into place along all or part of the edge of the container, and can be furthermore provided with a lateral tab with perforations on the upper part of said cover, for the opening of at least a part of the additional cover.

The subsequent removal of said additional cover causes the breakage of said teeth, so that the cover cannot be re-positioned or it is evident that it has been tampered with.

The attached drawings show a practical embodiment of the invention for illustrative non-limiting purposes.

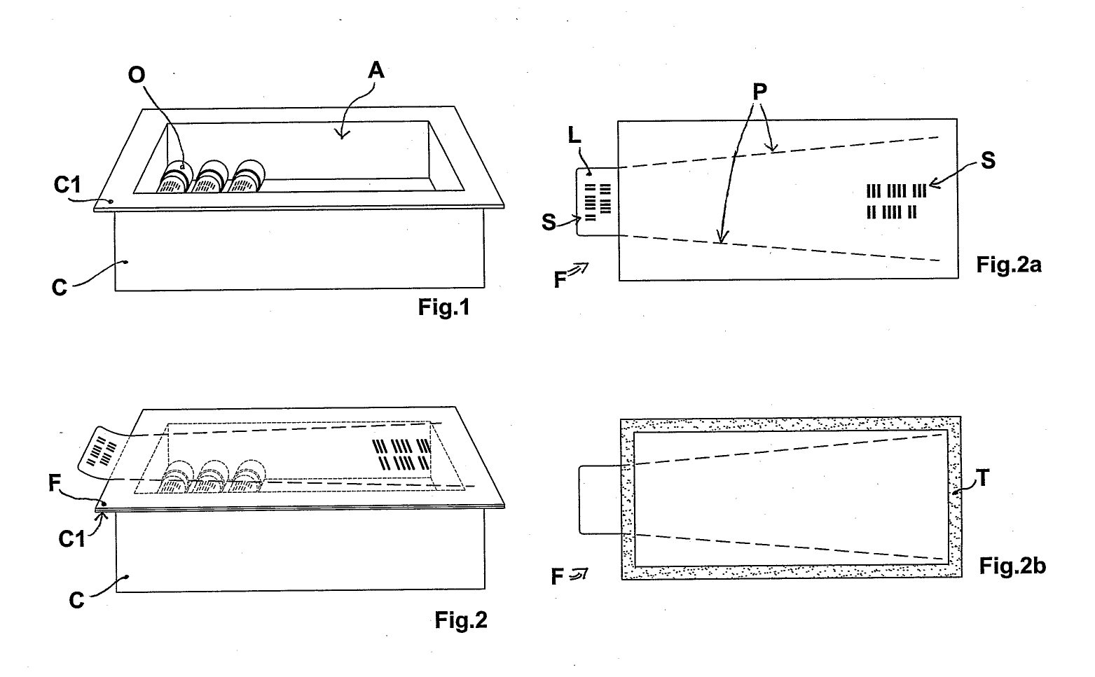

FIG. 1 shows the container, while FIG. 2 shows the safety patient kit completely closed and sealed.

In FIGS. 2a and 2b two views are shown, upper and lower, of the closing and identification element or sheet.

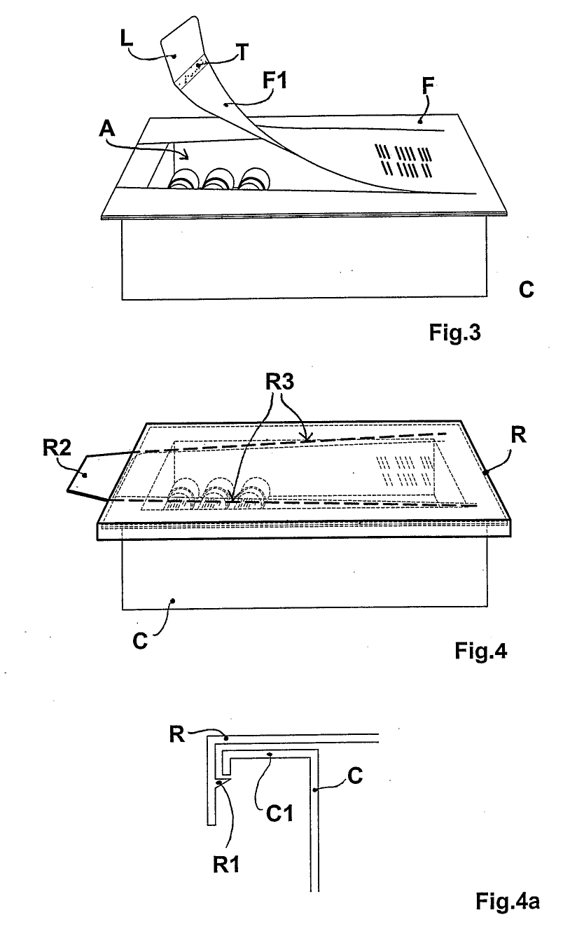

FIG. 3 shows the patient kit partially open, while FIG. 4 shows the container closed with the additional cover.

FIG. 4a schematises the closing method of the additional cover.

The new patient kit (K) comprises a container (C) having a parallelepiped, cubic or other shape and provided with at least one upper aperture (A) for the insertion of test tubes and/or vials (O) and/or auxiliary labels and/or other medical devices.

The patient kit (K) also comprises a closing and identification element (F), on which the identification data (S) of the patient and/or of the content and/or other information necessary for the person taking the sample are printed.

Said element (F) is an adhesive sheet wherein, for example, the adhesive layer (T) is distributed on the contact surface between said sheet (F) and the edge (C1) of said upper aperture (A) of the container (C).

Said closing element (F) comprises at least one tab (L), which can be provided with the identification data of the patient or of the content of the container, and one or more incisions or perforations (P) to permit opening of the sealed container.

In particular, said incisions or perforations (P) allow at least a part (F1) of said element (F) to be raised, giving access to the inside of the container (C).

After the opening of said closing element (F), it is possible to re-seal the container (C) via the use of an additional cover (R), detached from or integral with said container (C).

Said additional cover (R) is of the perimetric seal type, i.e. it comprises a plurality of teeth (R1) distributed along its perimeter, which snap into all or part of the edge (C1) of the container, preferably at the corners.

It can also have a lateral tab (R2) and perforations (R3) on the upper part, for tear-opening of at least part of the additional cover (R).

The re-opening of said additional cover (R) causes breakage of said teeth (R1).

Therefore, with reference to the preceding description and the attached drawings, the following claims are expressed.

1. Safety patient kit (K) for test tubes and/or vials (O) and/or other medical devices for use in hospitals, private laboratories, test points or treatment centres in general, characterised in that it comprises a container (C) with at least one aperture (A) for the insertion of said test tubes and/or vials (O) and/or medical devices in general, and at least one element (F) for closing and sealing said aperture (A), comprising one or more incisions or perforations (P) and/or other systems for tear-opening of at least a part (F1) of said element (F), for access inside said container (C), and wherein said element (F) is provided with the identification data (S), in plain text or in code, relating to the patient and/or the content and/or the use of the content and/or the origin and/or any other information necessary to the person taking the sample.

2. Safety patient kit (K) according to claim 1, characterised in that said closing and identification element (F) is an adhesive sheet comprising at least one protruding tab (L).

3. Safety patient kit (K) according to claims 1, 2, characterised in that said identification data are provided on any part of said element (F).

4. Safety patient kit (K) according to claims 1, 2, 3, characterised in that said closing and identification element (F) is printed in the phase prior or subsequent to its application to the container (C).

5. Safety patient kit (K) according to the preceding claims, characterised in that it is disposable.

6. Safety patient kit (K) according to the preceding claims, characterised in that it comprises a further cover (R), detached from or integral with said container (C), provided with a plurality of teeth (R) distributed along all or part of the perimeter of said cover (R) and designed to snap in along all or part of the edge (C1) of said container (C), and wherein the subsequent removal of said cover (R) causes the breakage of said teeth (R1).

7. Safety patient kit (K) according to the preceding claims, characterised in that said cover (R) comprises a lateral tab (R2) and/or perforations (R4) for tear-opening of at least a part of said additional cover (R).

8. Safety patient kit (K) according to the preceding claims, characterised in that it is positioned on a sliding belt and/or in another automatic device for the insertion of said test tubes and/or vials (O) and/or auxiliary labels and/or medical devices and/or for the application of said adhesive closing and identification sheet (F).
